(19) United States
(12) Patent Application Publication
(10) Pub. No.: US 2016/0038711 A1
(43) Pub. Date: Feb. 11, 2016

(54) STIMMING-REDUCTION VIEWING APERTURE AND METHOD OF USING

(71) Applicant: Jerry Perez, Miami, FL (US)

(72) Inventor: Jerry Perez, Miami, FL (US)

(21) Appl. No.: 14/452,693

(22) Filed: Aug. 6, 2014

(51) Int. Cl. A61M 21/00

(52) U.S. Cl. CPC A61M 21/0094

(57) ABSTRACT

An apparatus and method to reduce or eliminate stimming behavior by placing a viewing aperture in front of the user. This reduces visual stimulation by limiting what can be seen. After repeated use with the present invention and interactive mediums, new neural pathways are formed in the brain, which cause the reduction or elimination of stimming behavior.

### FIELD OF THE INVENTION

This invention is a way to reduce self stimulating behavior and an apparatus that is used to reduce self stimulating behavior. The purpose of the invention is to increase concentration in people, mainly children with disabilities such as autism.

### BACKGROUND OF THE INVENTION

Children with disabilities such as autism frequently engage in what is called self-stimulation, or stimming. Self-stimulation is a behavior classified by repeated physical movements or sounds. Therapists view this as a way in which these types of people calm themselves, and another common theory is that it is done to relieve emotions. Stimming often times gives kids a hard time socializing. Common stimming practices are, rocking, repeating noises or words, snapping fingers, hand flapping, etc.

The current invention is a set of goggles with the intended purpose of reducing self-stimulating behavior. It increases concentration on, writing, coloring puzzles, and other activates. The lenses of the goggles are interchangeable to comprise of either one or two lenses. The purpose of this is that in some people it is believed that the one of the sets of lenses provide more concentration benefits than the other, and the applicant may be tested on either the one lens set, or two lens set to determine which one helps the person more.

There exists a need to help kids with disabilities increase concentration.

There also exists a need to help relieve people of self-stimulatory behaviors, without harm of extreme measure or prescription medicine, non-prescription medicine, or supplements.

The current invention provides kids with attention disabilities such as self-stimulatory behaviors to increase concentration, and provide relief to some of their symptoms.

### SUMMARY OF THE INVENTION

One aspect of the present invention is a stimming-reduction viewing aperture, comprising: a single scope having an open proximal end and an open distal end; said single scope having a length; said single scope having a diameter; and a face attachment means connected to said single scope.

Another aspect of the present invention is a stimming-reduction viewing aperture, comprising: a dual scope having an open proximal end and an open distal end; said dual scope having a length; said dual scope having a diameter; and a face attachment means connected to said dual scope.

Another aspect of the present invention is a method of reducing stimming behavior, comprising the steps of: placing at least one of either a single scope, or a dual scope in front of the eye of the user; and placing an interactive medium to engage with the user.

### BRIEF DESCRIPTION OF THE DRAWINGS

FIG. 1 illustrates a pictorial of a front view of one embodiment of the present invention 10 on the head of the user;

FIG. 2 illustrates a pictorial view of the present invention on the head of the user;

FIG. 3 illustrates a pictorial view of a front view of another embodiment of the present invention;

FIG. 4 illustrates a pictorial view of the embodiment of FIG. 3 of the present invention;

FIG. 5 illustrates one embodiment of the present invention showing the base that the single lens attachment or dual lens attachment can interchangeably connect to; and

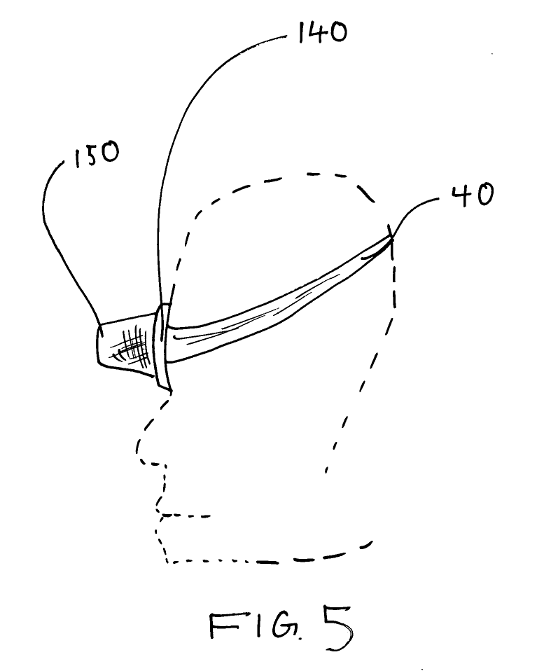

FIG. 6 illustrates one embodiment of how the invention may be used.

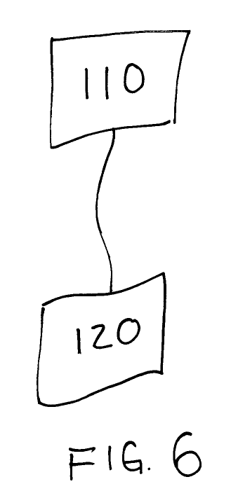

### REFERENCE NUMERALS LIST

5 Present invention or Stimming-Reduction Viewing Aperture and Method of Using
10 viewing aperture
20 single scope or aperture
30 dual scope or aperture
40 strap or face attachment means
50 interactive tool
100 interactive medium
110 placing at least one of either a single scope, or a dual scope in front of the eye of the user
120 step of placing an interactive medium in front of a user
130 diameter
140 proximal end
150 distal end

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Certain terminology will be used in the following description for convenience and reference only, and will not be limiting. For example, the words “upwardly,” “downwardly,” “rightwardly,” and “leftwardly” will refer to directions in the drawings to which reference is made. The words “inwardly” and “outwardly” will refer to directions toward and away from, respectively, the geometric center of the system and designated parts. Said terminology will include the words specifically mentioned, derivatives, and similar words. Also, “connected to,” “secured to,” or similar language includes the definitions “indirectly connected to,” “directly connected to,” “indirectly secured to,” and “directly secured to.”

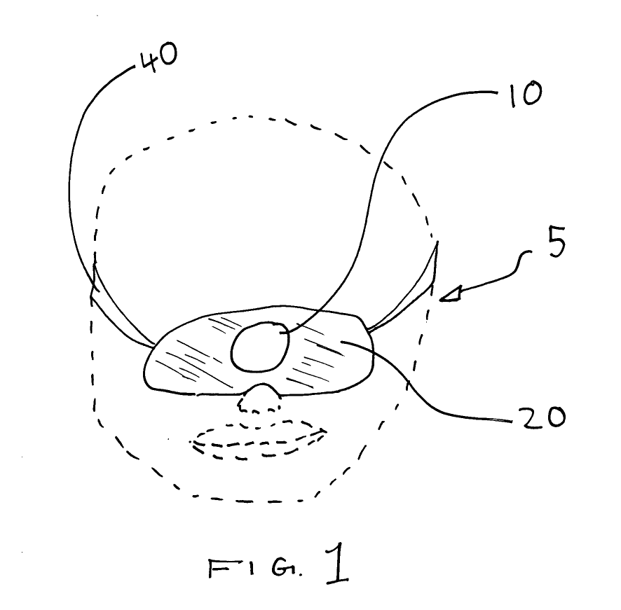

As illustrated in FIG. 1, one embodiment of the present invention 5 may have a single scope attachment 20. The present invention 5 may stay on the head of the user without using hands to hold the apparatus in place. The present invention 5 may be held in place by being connected to a hat, or a strap that extends around the head.

In other embodiments the single scope attachment 20 may have a diameter 130 that is less than 1 inch and greater than 2 inches. The present invention 5 may be referred to herein as stimming-reduction viewing aperture and method of using the stimming-reduction viewing aperture 5.

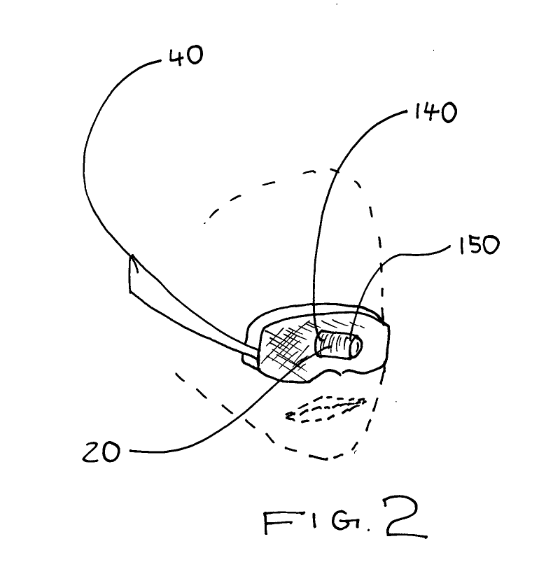

As illustrated of FIG. 2, the single scope attachment 20 may extend outwardly away from the user a distance from about 3 inches to 6 inches. In another embodiment the distance may be less than about 3 inches, or greater than 6 inches.

The single scope attachment 20 may have a cylindrical shaped housing with a diameter 130 of about 1 inch to 2 inches. The single scope attachment 20 may be open at both ends, with a proximal end 140 adjacent the face of the user so the user can see through the single scope attachment 20 and through the open distal end 150 to define one viewing aperture 10.

FIG. 2 also illustrates that the single scope attachment 20 may be removably attached or worn by the user via a strap 40 or head attachment means 40. The strap 40 may contain an elastic portion to allow it to fit a variety of head sizes and shapes.

The viewing aperture 10 may be open at both the proximal end 140 and distal end 150, or there may be a lens at either or both ends.

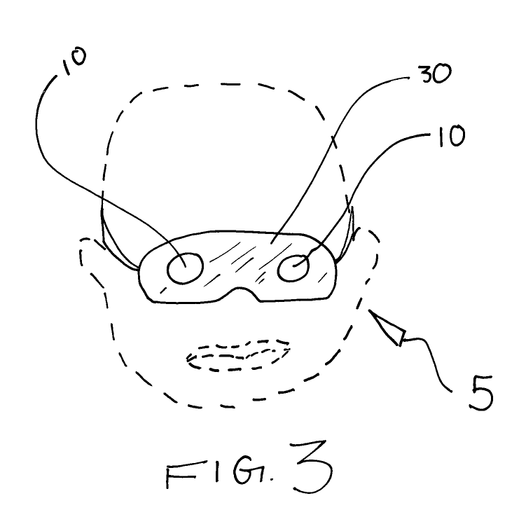

As illustrated in FIG. 3, another embodiment of the present invention 5 may have a dual scope attachment 30. The dual scope attachment 30 may extend outwardly away from the user a distance of about 3 to 6 inches. In another embodiment the distance may be less than about 3 inches, or greater than 6 inches. The dual scope attachment 30 may have two viewing apertures 10, one viewing aperture positioned over a left eye, and another viewing aperture 10 positioned over the right eye. In one embodiment the internal shape of the viewing aperture 10 is circular.

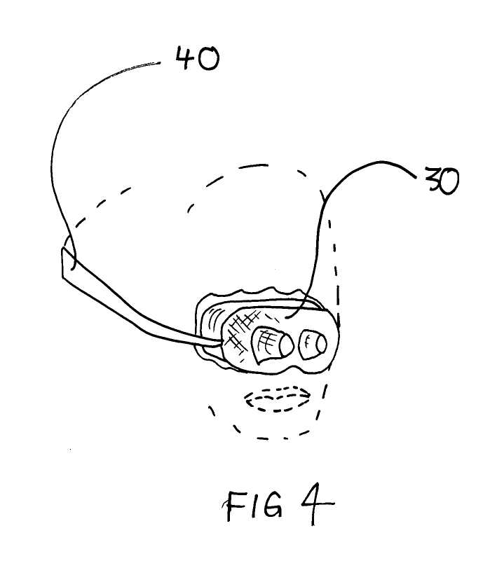

As illustrated in FIG. 4 the dual scope attachment 30 may extend outwardly away from the user a distance of about 2 to about 5 inches. In other embodiments the dual scope attachment 30 may extend outwardly away from the user a distance of less than 2 inches, or greater than 5 inches. The strap 40 or face attachment means 40 may removably connect to the dual scope attachment 30 and may be able to be elastically or non-elastically removably attached to the user. The dual scope attachment 30 may have two viewing apertures 10 that are opened and visible from a proximal end 140 to and through a distal end 150.

The present invention 5 may be used by placing the viewing aperture 10 of the single scope 30 so either eye, or both eyes can see through the viewing aperture 10.

In another embodiment the present invention 5 may be used by placing both viewing apertures 10 of the dual scope 30 in front of both eyes.

In either of the two above embodiments, the single scope 20 or dual scope 30 may be worn by the user for 30 minutes to two (2) hours. While wearing the single scope 20 or dual scope 30 the child may interact with an interaction medium 100. An interaction medium includes any device that the user or child is interacting with, but is not limited to adults or other sources such as a book, computer, monitor, hand held device, or television.

While the user is wearing the present invention 5, and engaging with an interactive medium 100, the child will experience less stimming behavior, which will form new neural pathways. Then after a number of sessions using the present invention 5, the reduced stimming behavior will remain with the child or user when the present invention 5 is not worn or used. Thus the user or child will behave with a heightened concentration, and behave with less non-typical behavior, such as less random body movements or twitches. This results in the child or user experiencing greater confidence, and more typical behavior.

When an interactive medium 100 placed in front of the user or child while wearing the present invention 5, their ability to focus is increased greatly, and there is very little stimming if any, unlike when the present invention 5 is not in coordination with the child or user.

For example, a video was recorded showing a child with self-stimulatory behavior listing to music from a device, the interactive medium 100 without wearing the present invention. The child was stimming and acting very uneasy, making non-typical movements with his jaw and lips, and hands.

However, immediately after wearing the present invention 5, his self-stimulatory behavior began to cease, and he calmly remained engaged with the music from the device, also referred to as the interactive medium 5.

In autistic children eye contact is scarcely engaged in. With the present invention being placed in front of the child or patient's eye, their ability to concentrate on the interactive medium 100 is much greater, and making eye contact is much easier.

For example another video display a child with obvious stimming behavior without use of the present invention 5. However when the present invention 5 is applied to the user, their ability to make eye contact and follow instructions was very good. Also the child or user was able to stay concentrated on a spot of focus like the eye. This behavior was not possible without use of the present invention 5.

The productive effect of improvement to kids with focus disabilities like ADD or ADHD as well as self stimulatory behavior found commonly in kids with autism by the current invention is undeniable, and the need for this item would be backed by the many families who seek a working means of relief for the said disabilities.

I claim:

1. A stimming-reduction viewing aperture (5), comprising:
a single scope (20) having an open proximal end (140) and an open distal end (150);
said single scope (20) having a length;
said single scope (20) having a diameter; and
a face attachment means (40) connected to said single scope (20).

2. A stimming-reduction viewing aperture (5), comprising:
a dual scope (30) having an open proximal end (140) and an open distal end (150);
said dual scope (30) having a length;
said dual scope (30) having a diameter; and
a face attachment means (40) connected to said dual scope (30).

3. A method of reducing stimming behavior, comprising the steps of:
placing at least one of either a single scope, or a dual scope in front of the eye of the user (110); and
placing an interactive medium to engage with the user (120).
